(19) United States
(12) Patent Application Publication
(10) Pub. No.: US 2008/0065060 A1
(43) Pub. Date: Mar. 13, 2008

(54) Controlled monopolar and bipolar application of RF energy

(76) Inventor: Moshe Ein-Gal, Ramat Hasharon (IL)

Correspondence Address: DEKEL PATENT LTD., DAVID KLEIN, BEIT HAROF'IM, 18 MENUHA VENAHALA STREET, ROOM 27, REHOVOT, 76209, omitted

(21) Appl. No.: 11/516,618

(22) Filed: Sep. 7, 2006

(51) Int. Cl. A61B 18/18

(52) U.S. Cl. 606/34; 606/38; 606/41

(57) ABSTRACT

A system including at least two target electrodes and at least one return electrode spaced outwards from the at least two target electrodes, at least two RF power sources in electrical communication with the electrodes and operative to generate RF energy waveforms to the at least two target electrodes, a controller in operative communication with the at least two RF power sources to control delivery of RF energy in a monopolar mode, a bipolar mode or a combined monopolar and bipolar mode, wherein in the monopolar mode at least one of the at least two target electrodes is energized by one of the RF power sources and cooperates with the at least one return electrode to deliver monopolar RF energy towards a first spatial region, and wherein in the bipolar mode the at least two target electrodes are energized by the at least two RF power sources to deliver bipolar RF energy between the at least two target electrodes in a second spatial region different from the first spatial region, and wherein in the combined monopolar and bipolar mode the at least two RF power sources deliver RF energy to the at least two target electrodes such that energy is delivered between the at least two target electrodes as well as between the at least two target electrodes and the at least one return electrode, and an actuator operative to cause relative motion between the at least two target electrodes and surrounding tissue.

### FIELD OF THE INVENTION

The present invention relates generally to electrosurgical apparatus for tissue ablation generally, and particularly to apparatus for radio frequency (RF) tissue ablation.

### BACKGROUND OF THE INVENTION

Radio frequency (RF) tissue ablation is a well-known technique, e.g., in electrosurgery and thermal therapy, for making thermal lesions in the vicinity of an uninsulated tip of an electrode due to tissue coagulation caused by resistive heating. Voltage applied to electrodes causes electrical current flow through tissue and heat production due to tissue electrical resistance (Joule heating). The electrode can be applied directly on superficial structures, surgically, endoscopically, laparascopically, or even via a transcatheter access such as a treatment for symptomatic cardiac arrhythmias. If the electrode is formed as a needle, then the electrode may be inserted interstitially, and guided by imaging.

In a monopolar mode, current flows between a small target electrode and a large counter-electrode placed further away from the target. Due to the difference in the sizes of the electrodes, current density and associated Joule heat production are much higher at the target than at the return electrode. In contrast, in a bipolar mode, high density current flows between two adjacent target electrodes. Joule heat production is confined to a small volume due to electrodes size and proximity.

Thermal treatment amounts to applying high density current for a sufficient time to cause elevated temperature and associated physiological changes, e.g., coagulation, at a volume of tissue. Monopolar current flows through a larger volume compared to bipolar current. Consequently, monopolar Joule heating has a deeper penetration compared to bipolar heating, where the heat is confined to a small volume at the target electrodes

Electrosurgical apparatus is known that provides an option of selecting and switching between pure monopolar mode and pure bipolar mode. For example, U.S. Pat. No. 6,837,884 to Woloszko describes electrosurgical apparatus and methods for ablating, coagulating, shrinking, stiffening, or other treatment of a target tissue of a patient. The apparatus includes an electrosurgical probe, and an introducer needle adapted for passing through the distal end of the probe. In some embodiments, the electrosurgical system may include a dispersive return electrode for switching between bipolar and monopolar modes.

### SUMMARY OF THE INVENTION

The present invention seeks to provide a novel system for application of RF energy that combines monopolar and bipolar modes of operation, as is described more in detail hereinbelow.

There is provided in accordance with an embodiment of the present invention a system including at least two target electrodes and at least one return electrode spaced outwards from the at least two target electrodes, at least two RF power sources in electrical communication with the electrodes and operative to generate RF energy waveforms to the at least two target electrodes, an actuator operative to cause relative motion between the at least two target electrodes and surrounding tissue, and a controller in operative communication with the at least two RF power sources to control delivery of RF energy in a monopolar mode, a bipolar mode or a combined monopolar and bipolar mode, wherein in the monopolar mode at least one of the at least two target electrodes is energized by one of the RF power sources and cooperates with the at least one return electrode to deliver monopolar RF energy towards a first spatial region, and wherein in the bipolar mode the at least two target electrodes are energized by the at least two RF power sources to deliver generally bipolar RF energy between the at least two target electrodes in a second spatial region different from the first spatial region, and wherein in the combined monopolar and bipolar mode the at least two RF power sources deliver RF energy to the at least two target electrodes such that energy is delivered between the at least two target electrodes as well as between the at least two target electrodes and the at least one return electrode. The selection of monopolar mode, bipolar mode or combined mode may be determined by the actuator status, that is, by the relative motion of the at least two target electrodes with respect to the surrounding tissue.

Each target electrode may have its own return electrode. Alternatively, the at least one return electrode may serve as a common return electrode to all the target electrodes.

The actuator causes relative motion between the target electrodes and the surrounding tissue, wherein the relative positions of the at least two target electrodes to each other may be fixed or may change. For example, the target electrodes may be moved with respect to the surrounding tissue, but the target electrodes do not move with respect to each other As another example, the actuator may cause relative motion between the target electrodes such that one of the target electrodes penetrates deeper into tissue than another of the target electrodes, such that as the target electrodes are moved with respect to the surrounding tissue, the target electrodes move with respect to each other, too.

In accordance with a non-limiting embodiment of the present invention, the target electrodes may be helical in shape, and they may or may not be concentric with each other. The target electrodes may be insulated except for an active tip portion which is operative to deliver RF energy. The actuator may simultaneously rotate the target electrodes in a corkscrew manner such that the inter-electrode spacing is fixed and the controller may control the combined mode of RF energy delivery according to the actuator status (i.e., the positions of the electrodes).

In accordance with a non-limiting embodiment of the present invention, the inter-electrode spacing changes according to the actuator status, i.e., according to the relative position of the electrodes with respect to the surrounding tissue.

In accordance with a non-limiting embodiment of the present invention, the target electrodes may be non-concentrically helical in shape. The target electrodes may be insulated except for an active tip portion which is operative to deliver RF energy. The actuator may simultaneously rotate the target electrodes in a corkscrew manner such that the inter-electrode spacing changes according to the actuator status (i.e., the positions of the electrodes).

### BRIEF DESCRIPTION OF THE DRAWINGS

The present invention will be understood and appreciated more fully from the following detailed description taken in conjunction with the drawings in which:

FIG. 1 is a simplified illustration of a system for controlling monopolar and bipolar modes of RF energy delivery, constructed and operative in accordance with an embodiment of the present invention;

FIGS. 2A and 2B are simplified top-view illustrations of target electrodes of the system of FIG. 1 constructed as helical electrodes, wherein as the target electrodes rotate, a distance between the active tip portions of the electrodes changes; and

FIG. 2C is a simplified illustration of the target electrodes of the system of FIG. 1 with one of them penetrating deeper into tissue than another of them.

### DETAILED DESCRIPTION OF EMBODIMENTS

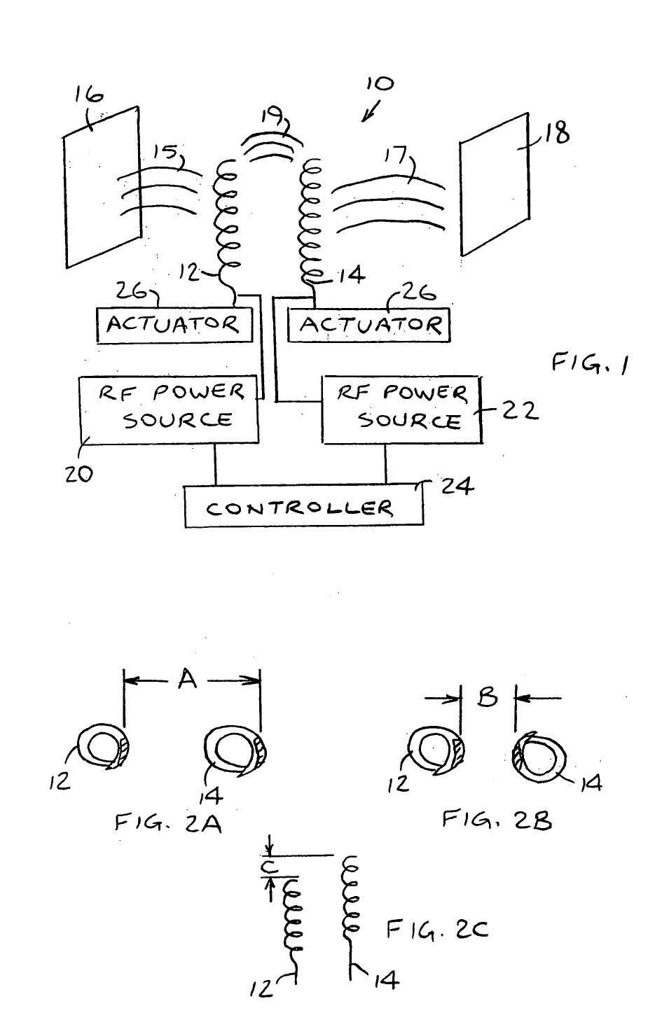

Reference is made to FIG. 1, which illustrates a system 10 for controlling monopolar and bipolar modes of RF energy delivery, constructed and operative in accordance with an embodiment of the present invention.

System 10 may include two or more target electrodes 12 and 14 (two are shown in the non-limiting illustrated embodiment) and one or more return electrodes (two are shown in the non-limiting illustrated embodiment, return electrodes 16 and 18). RF power sources 20 and 22 may energize the target electrodes 12 and 14 (the return electrodes 16 and 18 may be grounded or may also be energized to some level by RF power sources) by generating RF energy waveforms (e.g., voltage or current waveforms) to the electrodes. A controller 24 may control and manipulate the waveforms to the electrodes. The controller 24 may include any known device for varying operative characteristics of the RF energy generated by the RF power sources 20 and 22, such as but not limited to, frequency, phase and amplitude, and may control the timing and duration of the delivery of RF energy to the electrodes. In such a manner, system 10 is capable of selectively combining monopolar and bipolar modes to better control the current distribution in a target (e.g., tissue in a human body or non-human body or inanimate object).

The target electrode 12 paired with the return electrode 16 defines a monopolar channel to deliver monopolar RF energy towards a spatial region indicated by numeral 15. Similarly, target electrode 14 paired with return electrode 18 defines a monopolar channel to deliver monopolar RF energy towards a spatial region indicated by numeral 17. Energizing of both target electrodes 12 and 14 creates a bipolar mode of energy delivery between them in a spatial region (indicated by numeral 19) different from the monopolar spatial regions. In the combined monopolar and bipolar mode, the RF power sources 20 and 22 deliver RF energy to the target electrodes 12 and 14 such that energy is delivered between the target electrodes 12 and 14 as well as between the target electrodes 12 and 14 and the return electrodes 16 and 18.

Each target electrode may have its own return electrode. Alternatively, the return electrode may serve as a common return electrode to all the target electrodes.

The target electrodes 12 and 14 may be constructed of any shape, such as but not limited to, straight and pointed or blunt, curved, bent or helical. In the non-limiting illustrated embodiment the target electrodes are helical (corkscrew) in shape.

An actuator 26 (such as a step motor, servomotor, linear actuator, and the like) may be provided to cause relative motion between the target electrodes 12 and 14 and surrounding tissue. In the non-limiting illustrated embodiment, there are two separate and dedicated actuators 26 for each target electrode 12 and 14, such as servomotors that rotate the target electrodes 12 and 14. In this manner, the actuators 26 may cause relative motion between the target electrodes 12 and 14 themselves. In other words, the actuators 26 may cause relative motion between the target electrodes 12 and 14 and the surrounding tissue, wherein the relative positions of the at least two target electrodes 12 and 14 to each other may be fixed or may change. The actuators 26 may operate in a closed control loop with sensors (not shown) that sense a temperature or resistance or other parameter of the ablated tissue. Based on the sensed parameter, the actuators 26 may be controlled (e.g. by means of controller 24 or other controller) to move the target electrodes 12 and 14 to other portions of tissue. The selection of monopolar mode, bipolar mode or combined mode by controller 24 may be determined by the actuator status, that is, by the relative motion of the target electrodes 12 and 14 with respect to the surrounding tissue.

For example, as seen in FIGS. 2A and 2B, target electrodes 12 and 14 may be insulated except for an active tip portion 28 which is operative to deliver RF energy. The active tip portions 28 may extend over a partial peripheral portion of the target electrodes 12 and 14. As seen in FIGS. 2A and 2B, as the target electrodes 12 and 14 rotate, a distance between the active tip portions 28 changes (from distance A to distance B, wherein A>B). Additionally, for example, the actuators 26 may adjust the distance between the target electrodes 12 and 14 to be smaller than the spacing between the target electrodes 12, 14 and the return electrodes 16, 18. As another example, as seen in FIG. 2C, the actuators 26 may cause relative motion between the target electrodes 12 and 14 such that one of the target electrodes penetrates deeper into tissue than another of the target electrodes (as indicated by distance C).

As another example, the target electrodes 12 and 14 may be helical and concentric with each other. The target electrodes 12 and 14 may be insulated except for the active tip portions 28 which are operative to deliver RF energy. The actuator 26 may simultaneously rotate the target electrodes 12 and 14 in a corkscrew manner such that the inter-electrode spacing is fixed and the controller 24 may control the combined mode of RF energy delivery according to the actuator status (i.e., the positions of the electrodes 12 and 14). In accordance with a non-limiting embodiment of the present invention, the inter-electrode spacing may change according to the actuator status, i.e., according to the relative position of the electrodes 12 and 14 with respect to the surrounding tissue.

Thus the invention may be used to create varying and adjustable regions of RF energy delivery heretofore not possible with prior art systems.

The scope of the present invention includes both combinations and subcombinations of the features described hereinabove as well as modifications and variations thereof which would occur to a person of skill in the art upon reading the foregoing description and which are not in the prior art.

What is claimed is:

1. A system comprising:
at least two target electrodes and at least one return electrode spaced outwards from said at least two target electrodes;
at least two RF power sources in electrical communication with said electrodes and operative to generate RF energy waveforms to said at least two target electrodes;
a controller in operative communication with said at least two RF power sources to control delivery of RF energy in a monopolar mode, a bipolar mode or a combined monopolar and bipolar mode, wherein in said monopolar mode at least one of said at least two target electrodes is energized by one of said RF power sources and cooperates with said at least one return electrode to deliver monopolar RF energy towards a first spatial region, and wherein in said bipolar mode said at least two target electrodes are energized by said at least two RF power sources to deliver bipolar RF energy between said at least two target electrodes in a second spatial region different from said first spatial region, and wherein in the combined monopolar and bipolar mode the at least two RF power sources deliver RF energy to the at least two target electrodes such that energy is delivered between the at least two target electrodes as well as between the at least two target electrodes and the at least one return electrode; and
an actuator operative to cause relative motion between said at least two target electrodes and surrounding tissue.

2. The system according to claim 1, wherein said controller is operative to choose between the monopolar mode, bipolar mode and the combined monopolar and bipolar mode by a sensed relative motion of said at least two target electrodes with respect to the surrounding tissue.

3. The system according to claim 1, wherein a spacing between said at least two target electrodes does not change during relative motion of said at least two target electrodes with respect to the surrounding tissue.

4. The system according to claim 1, wherein a spacing between said at least two target electrodes changes during relative motion of said at least two target electrodes with respect to the surrounding tissue.

5. The system according to claim 1, wherein each target electrode has its own return electrode.

6. The system according to claim 1, wherein said at least one return electrode serves as a common return electrode to all said target electrodes.

7. The system according to claim 1, wherein said actuator is operative not only to cause relative motion between said at least two target electrodes and the surrounding tissue but said actuator is also operative to cause relative motion between said target electrodes.

8. The system according to claim 1, wherein said actuator causes relative motion between said target electrodes such that one of the target electrodes penetrates deeper into tissue than another of the target electrodes.

9. The system according to claim 1, wherein said target electrodes are helical in shape.

10. The system according to claim 9, wherein said target electrodes are insulated except for an active tip portion which is operative to deliver RF energy.

11. The system according to claim 10, wherein said actuator is operative to rotate said target electrodes in a corkscrew manner.

12. The system according to claim 11, wherein said active tip portions extend over a partial peripheral portion of said target electrodes, such that as said target electrodes rotate, a distance between said active tip portions changes.
